(19) United States
(12) Patent Application Publication
(10) Pub. No.: US 2025/0051838 A1
(43) Pub. Date: Feb. 13, 2025

(54) METHOD FOR DETECTING TARGET SEQUENCES

(71) Applicant: 3CR BIOSCIENCE LTD, Harlow (GB)

(72) Inventor: Nisha JAIN, Harlow (GB)

(73) Assignee: 3CR BIOSCIENCE LTD, Harlow (GB)

(21) Appl. No.: 18/722,768

(22) PCT Filed: Dec. 23, 2022
(86) PCT No.: PCT/GB2022/053386

§ 371 Date: Jun. 21, 2024

(30) Foreign Application Priority Data
Dec. 29, 2021 (GB) 2119080.6

(51) Int. Cl. C12Q 1/6853

(52) U.S. Cl. CPC C12Q 1/6853

(57) ABSTRACT

The present invention relates to a method for detecting one or more target sequences in a sample by amplification. Kits and compositions for use with the method are also provided.

### FIELD OF THE INVENTION

The present invention relates to methods and kits for nucleic acid detection in an assay system.

### BACKGROUND OF THE INVENTION

Advancements in molecular biology techniques, in particular the polymerase chain reaction (PCR) has enabled the development of processes which enable a greater understanding of an individual's genetic make-up (i.e. their genotype).

Genotyping is a generic term given to various nucleic acid analysis techniques that identify alterations or polymorphisms within known sequences, and usually for a given individual. These techniques are useful in many aspects of scientific, medical and forensic fields. For example, these techniques can be used to examine particular genetic loci in an individual in order to diagnose hereditary diseases or provide prognosis based on known genetic lesions. Particular modifications of interest include, for example, point mutations, deletions, insertions and inversions as well as polymorphisms within nucleic acid sequences of interest. These techniques can also be used for clinical purposes such as tissue typing for histocompatibility or for forensic purposes such as identity or paternity determination.

In many PCR based genotyping reactions, a signal producing system is employed to detect the production of amplified product. One type of signal producing system that is commonly used is the fluorescence energy transfer (FRET) system, in which a nucleic acid detector includes fluorescence donor and acceptor groups (see, for example, European Patent 1726664). A primary consideration with PCR-based techniques that employ a FRET-based system is that the signal generated must be highly specific and sensitive to ensure accurate results. High fidelity amplification is also critical.

A universal polymorphism detection system was described in WO 2020/144480.

However, there remains a need to be able to detect polymorphisms in samples which first require a step of reverse transcription—i.e. if the sample partly or wholly comprises RNA which. While reverse transcription can be performed as a separate step, this can be cumbersome and it would be preferred to perform genotyping reactions as part of a “one-step” reaction, where the reverse transcriptase is included in the PCR amplification mix. A one-step method is preferable for a number of reasons, including:
Being physically easier to carry out as no sample manipulation is required beyond the original arraying;
Faster than two-step methods;
Reduces the risk of sample cross-contamination which may happen between steps;
Reduces waste.

However, one-step reactions associated with its own difficulties given the different buffer conditions required for the reverse transcriptase (RT) to operate in (when compared to e.g. the DNA polymerase).

Thus, there is a continuing need to improve genotyping systems which require a step of reverse transcription, and in particular one-step RT genotyping systems.

### SUMMARY OF THE INVENTION

In one aspect, the present invention provides a method for detecting one or more target sequences in a sample comprising RNA by amplification, the method comprising:
a) providing an aqueous composition comprising:
   i) one or more forward oligonucleotide primers having a 5′ region which is a tag sequence and a 3′ region specific for a target sequence;
   ii) a reverse oligonucleotide primer, such that a forward and the reverse primer operably form a primer pair;
   iii) one or more probes having a reporter label and a quencher label, each probe comprising a sequence having at least 50% identity to the tag sequence of a forward oligonucleotide primer; and
   iv) optionally one or more enhancer oligonucleotide primers comprising a sequence having at least 50% identity to the sequence of a probe;
b) combining the primers, the probes, the sample, a polymerase, and a reverse transcriptase in a reaction mixture with a buffer; and
c) performing a one-step polymerase chain reaction (PCR) on the reaction mixture to generate tagged nucleic acids, whereby the probe(s) can bind the tagged nucleic acids.

In another aspect, the present invention also provides a kit for use in a nucleic acid amplification process, comprising:
i) one or more forward oligonucleotide primers having a 5′ region which is a tag sequence and a 3′ region specific for a target sequence;
ii) a reverse oligonucleotide primer, such that a forward and the reverse primer operably form a primer pair;
iii) one or more probes having a reporter label and a quencher label, each probe comprising a sequence having at least 50% identity to the tag sequence of a forward oligonucleotide primer;
iv) a polymerase;
v) a reverse transcriptase;
vi) a buffer; and
vii) optionally one or more enhancer oligonucleotide primers comprising a sequence having at least 50% identity to the sequence of a probe.

In another aspect, the present invention also provides a buffer composition suitable for a one-step reverse transcriptase polymerase chain reaction (RT-PCR), wherein the buffer comprises:
(a) KCl and the KCl is at a working concentration of no more than 60 mM, optionally wherein the working concentration of KCl is about 40 mM to about 60 mM, about 45 mM to about 55 mM, or about 50 mM; and
(b) a non-ionic surfactant.

In some embodiments, the buffer comprises KCl. The KCl may be at a working concentration of no more than 60 mM, optionally wherein the working concentration of KCl is about 40 mM to about 60 mM, about 45 mM to about 55 mM, or about 50 mM.

In some embodiments, the buffer comprises a non-ionic surfactant. Non-ionic surfactants are known in the art. Preferably, the non-ionic surfactant is selected from Tergitol™ Triton, or Igepal. In some embodiments, the non-ionic surfactant is selected from Tergitol 15-S-9, Tergitol 15-S-40, Tergitol 15-S-3, Triton X-100 or Igepal, optionally wherein the non-ionic surfactant is Tergitol 15-S-40.

The non-ionic surfactant may be present at a working concentration of at least about 0.01%, optionally wherein the non-ionic surfactant is present at a working concentration of about 0.05%.

In some embodiments, The buffer may further comprises one or more of:
i) Tris-HCl, preferably Tris-HCl at pH8.3;
ii) MgCl;
iii) Glycerol;
iv) TMAC; and/or
v) an RNase inhibitor.

In some embodiments, the reporter label may be located at a different portion of the probe compared to the quencher label. In some embodiments, the position of the labels are such that when free in solution, the reporter label and quencher label come into close proximity to each other such that little or no emission can be detected from the reporter label.

In some embodiments, the polymerase is a DNA polymerase. In some embodiments, the polymerase is a fragment or domain or a derivative of Taq polymerase. In some embodiments, the polymerase is a modified polymerase. In some embodiments, the polymerase is modified to reduce and/or remove exo-nuclease activity and/or modified to be a “hot-start” polymerase.

In some embodiments, the one or more probe(s) comprise a sequence having at least 60%, 70%, 80%, 90%, 95%, 98% or 99% identity to the tag sequence of a (forward) oligonucleotide primer. In some embodiments, the one or more probe(s) comprise a sequence identical to the tag sequence of a (forward) oligonucleotide primer.

In some embodiments, the one or more enhancer oligonucleotide primers comprise a sequence having at least 60%, 70%, 80%, 90%, 95%, 98% or 99% identity to the sequence of a probe.

In some embodiments, the one or more enhancer oligonucleotide primer(s) comprise a sequence identical to the sequence of a probe.

In some embodiments of the methods of the invention, at least 4, 10, 15, 20, 25 or 30 cycles of PCR are performed. In some embodiments, no more than 35, 40, 45 or 50 cycles of PCR are performed.

In some embodiments o the methods of the invention, the one-step polymerase chain reaction (PCR) step comprises a reverse transcription step prior to the PCR cycles.

In some embodiments of the methods of the invention, the method may further comprise:
d) measuring the signal generated by the binding of the probes to the tagged nucleic acids. The measurement may be made in real time or at the end of the reaction.

In some embodiments of the methods of the invention, the total volume of the reaction mixture is under 1 μL.

Compositions and kits for use with the methods described herein are also provided.

### BRIEF DESCRIPTION OF THE FIGURES

FIG. 1—Data showing the endpoint fluorescence detection for a one-step RT PCR assay using three different buffer solutions as part of the reaction mixture to detect single nucleotide polymorphisms in an RNA sample. The sequences of the primers and probes, as well as the composition of the buffer solutions can be found in Example 1. The data obtained was then plotted as FAM signal divided by ROX on the X axis, and HEX signal divided by ROX on the Y axis (the signals are normalised by ROX). Three discernible groups associated with respective genotypes were observed only in the condition using Buffer 1—the optimised buffer composition described herein. Neither buffer 2 or buffer 3 showed any amplification or signal.

FIG. 2—Data showing the endpoint fluorescence detection for a one-step RT PCR assay to detect a variant allele for SARS-CoV-2. This assay also used an enhancer primer. The assay system utilises three unlabelled oligonucleotide primers and two dual-labelled probes. The data obtained was then plotted as FAM signal divided by ROX on the X-axis, and HEX signal divided by ROX on the Y-axis (the signals are normalised by ROX). Three discernible groups associated with respective genotypes were observed.

FIG. 3—Real time signal detection for a one-step RT PCR assay for a sample which was serially diluted to show detection for varying RNA copies/μl. Fluorescence was recorded in real-time at each PCR cycle during the annealing and extension step. The resulting data was plotted as delta Rn vs cycle number and Ct values were calculated automatically using ABI 7900 software.

FIG. 4—Data showing the endpoint fluorescence detection for a one-step RT PCR assay using three different KCl concentrations as part of the buffer in the reaction mixture to detect single nucleotide polymorphisms in an RNA sample. The data obtained was then plotted as FAM signal divided by ROX on the X axis, and HEX signal divided by ROX on the Y axis (the signals are normalised by ROX). Three clearly discernible groups associated with respective genotypes were observed only in the condition using 100 mM KCl (in the 2× Buffer). Amplification was diminished in increasing KCl concentrations.

FIG. 5—Data showing the endpoint fluorescence detection for a one-step RT PCR assay in the presence and absence of a non-ionic surfactant as part of the buffer in the reaction mixture to detect single nucleotide polymorphisms in an RNA sample. The data obtained was then plotted as FAM signal divided by ROX on the X axis, and HEX signal divided by ROX on the Y axis (the signals are normalised by ROX). Three clearly discernible groups associated with respective genotypes were observed only in the condition which comprised a non-ionic surfactant. Amplification was diminished in the condition where a non-ionic surfactant was not present.

FIG. 6—Schematic of the universal polymorphism detection system methods as employed herein.

### DETAILED DESCRIPTION

These aspects relate to a new one-step reverse transcriptase (RT) PCR assay, which can be used as part of methods for detecting a target sequence in a sample comprising RNA. The method can also be employed as a UPDS (Universal polymorphism detection system) for samples comprising RNA. As such, the methods of the invention use a reverse transcriptase. Moreover, the methods of the invention also utilise a particular buffer, which is part of the resulting reaction mixture. The buffers described herein are optimised for the one-step RT PCR assays of the invention.

The assays described herein may, when used for the purposes of detecting variants and/or alleles, comprise the use of two (or more, for example, three or four or five or more) competing allele-specific oligonucleotide primers. Each may comprise a portion of template sequence at the 3′ end (which may differentiate between the alleles) and different non-template sequence tails, or tags, to the 5′ end, and a (common) reverse primer. This system can use a reporting system, such as a fluorescent reporting system. This system may use a probe, such as an oligonucleotide probe. This system can then bind to the complementary sequence introduced by each of the tails of the allele-specific primers, leading to generation of detectable signal, such as a light signal.

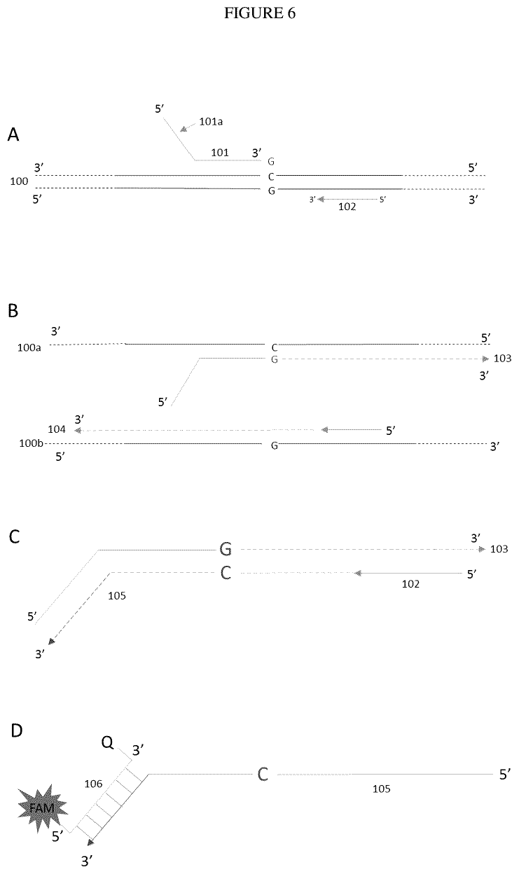

A schematic of an exemplary UPDS process which may be used as part of the present invention is shown in FIG. 6 In step (A), the correct allele specific primer 101 (only one shown for simplicity) with tail/tag 101a and reverse primer 102 are shown alongside the template DNA 100, showing the single nucleotide polymorphism (SNP) which exemplifies the specific allele (here it is a C-G). Following denaturation (into single stranded templates 100a and 100b), the primers anneal and extend, and step (B) shows the resulting synthesis of two new strands 103 and 104. One of these strands 103 is tailed/tagged as a result of the use of the allele-specific primer, and subsequent extension steps—for example step (C)—causes synthesis of a strand comprising the complement of the allele-specific primer tail/tag 105. In step (D), this shows that the generation of the complement of the allele-specific tail/tag allows a probe such as the exemplified dual-labelled probe 106 (with a FAM fluorophore and a quencher) to bind/base-pair to the tail/tag portion and generate a detectable signal.

The methods described herein may comprise the presence of an enhancer primer. The enhancer primer as disclosed herein may comprise a sequence having at least 50% identity (e.g. at least 60%, 70%, 80%, 90%, 95%, 98% or 99% identity) to the sequence of a probe and thus is also capable of binding to the tag sequence. Here, following step (C) from FIG. 6, the enhancer primer is able to compete with the probe for binding to the allele-specific tail/tag of synthesised strand 105. This allows for further rounds of amplification to generate further strands 105 resulting in a greater number of probe binding sites.

In one aspect, the present invention provides a method for detecting one or more target sequences in a sample comprising RNA by amplification, the method comprising:
a) providing an aqueous composition comprising:
   i) one or more forward oligonucleotide primers having a 5′ region which is a tag sequence and a 3′ region specific for a target sequence;
   ii) a reverse oligonucleotide primer, such that a forward and the reverse primer operably form a primer pair;
   iii) one or more probes having a reporter label and a quencher label, each probe comprising a sequence having at least 50% identity to the tag sequence of a forward oligonucleotide primer; and
   iv) optionally one or more enhancer oligonucleotide primers comprising a sequence having at least 50% identity to the sequence of a probe;
b) combining the primers, the probes, the sample, a polymerase, and a reverse transcriptase in a reaction mixture with a buffer; and
c) performing a one-step polymerase chain reaction (PCR) on the reaction mixture to generate tagged nucleic acids, whereby the probe(s) can bind the tagged nucleic acids.

In another aspect, the present invention also provides a kit for use in a nucleic acid amplification process, comprising:
i) one or more forward oligonucleotide primers having a 5′ region which is a tag sequence and a 3′ region specific for a target sequence;
ii) a reverse oligonucleotide primer, such that a forward and the reverse primer operably form a primer pair;
iii) one or more probes having a reporter label and a quencher label, each probe comprising a sequence having at least 50% identity to the tag sequence of a forward oligonucleotide primer;
iv) a polymerase;
v) a reverse transcriptase;
vi) a buffer; and
vii) optionally one or more enhancer oligonucleotide primers comprising a sequence having at least 50% identity to the sequence of a probe.

In another aspect, the present invention also provides a buffer composition suitable for a one-step reverse transcriptase polymerase chain reaction (RT-PCR), wherein the buffer comprises:
(a) KCl and the KCl is at a working concentration of no more than 60 mM, optionally wherein the working concentration of KCl is about 40 mM to about 60 mM, about 45 mM to about 55 mM, or about 50 mM; and
(b) a non-ionic surfactant.

The term “sample” as used herein should be understood to mean a sample, which generally comprises or consists of RNA. In some embodiments, the sample comprises RNA. Generally, the sample may be a biological sample, for example a biological fluid selected from blood or a blood derived product such as plasma or serum, saliva, urine, sweat, or cerebrospinal fluid. The sample may also be a sample of cells or tissue, for example muscle, nail, hair, bone, marrow, brain, vascular tissue, kidney, liver, peripheral nerve tissue, skin and epithelial tissue. The tissue or cells may be normal or pathological tissue. Generally, the sample may be treated to remove all material except RNA by techniques well known in the art. PCR sample preparation protocols are also well described in the literature and are available from the websites of Agilent, Life Technologies, Qiagen and Illumina. In one embodiment, the method of the invention may be employed to determine the presence of a particular allele of a locus, for example the presence of a SNP in a gene or other locus in RNA. In some embodiments, the sample comprises RNA of unknown origin and the method of the invention may be employed to detect the presence of a target RNA in the sample. The RNA present in a sample may be referred to as the RNA template in the context of the present disclosure.

The term “primer” as used herein refers to a synthetically or biologically produced single-stranded oligonucleotide. In use, primers can typically base pair/anneal to another single-stranded nucleic acid molecule using the rules defined by Watson-Crick base pairing to form a double-stranded nucleic acid duplex, whereby the primer strand can then be extended/elongated with an appropriate polymerase in the presence of nucleotide monomers. It is well known that many such nucleic acid polymerases or reverse transcriptases require the presence of a primer that may be extended to initiate such nucleic acid synthesis. For example, the oligonucleotides disclosed herein may be used as one or more primers in various extension, synthesis, or amplification reactions. It is also well known that in PCR assays, primers usually exist in primer pairs, which comprises of a nominal “forward” primer and a nominal “reverse” primer. Forward and reverse primers may be differentiated by the fact that they bind to different strands of a given duplex template, and operably form a primer pair such that the reverse primer binds downstream to the forward primer on the nucleic acid duplex/template (i.e. in the 3′ direction of the forward primer).

The term “portion” as used herein with reference to oligonucleotide constructs (such as primers) should be understood to mean a contiguous part of said oligonucleotide construct which comprises at least one, two, three or more contiguous bases.

In some embodiments, primers may have at least one portion which is specific for a target sequence (i.e. a sequence-specific primer). In other words, primers may be designed in such a way that at least one portion will have a sequence which is complementary to a target known sequence which is to be detected. Thus, in a given amplification reaction, a sequence-specific primer will preferentially base-pair/anneal to its target sequence.

Sequence-specific primers may also be allele-specific primers whereby the target sequence is a specific allele at a genetic locus which can be used to alleles that share a common polymorphism. The term “allele” as used herein refers to a genetic variation associated with a gene or a segment of DNA, i.e. one of two or more alternate forms of a DNA sequence occupying the same genetic locus. In other words, allele-specific primers may be used to amplify a single allele and may be capable of differentiating between two sequences that differ by only a single base difference of change. The difference between the two alleles may be a SNP, an insertion, or a deletion.

Sequence-specific primers may be universal for all alleles. In other words, sequence-specific primers may only be specific for a locus which is universal for all alleles and/or polymorphisms to be detected. These primers can therefore be used as universal primers. In other words, one universal sequence-specific primer can be used in conjunction with two or more allele-specific primers in a method of the invention as described herein. Thus, in one embodiment of the method of the invention, the one or more forward oligonucleotide primers may be allele-specific primers which have a sequence-specific portion which is specific for one or more known allele(s). In such embodiments, the reverse oligonucleotide primers may be sequence-specific primers specific for a known sequence near the allele. In one example, the sequence bound by the reverse primer is known to not be polymorphic and/or not be allele dependent.

Sequence-specific and/or allele-specific primers may also comprise a 5′ portion which is a tag sequence, wherein tag sequence is not complementary to the target sequence or allele. This organisation allows for a tag sequence to be incorporated as part of the primer during a specific polymerisation reaction. Subsequent cycles during a PCR reaction will then allow the tag sequence to be amplified further and become incorporated as part of the amplified duplex. Tag sequences may be useful as they can be designed to be specific for complementary probe sequences, whereby the probes can be labelled using a reporter label to allow for the generation of an associated signal in response to amplification from a sequence-specific and/or allele-specific primer.

In some embodiments, the sequence of the sequence-specific portion of target-specific primers as used herein, such as allele-specific primers, can be designed such they are at least 50%, 60%, 70%, 80%, 85%, 90%, 95%, 96%, 97%, 98%, 99% or 100% identity to their target sequences (for example, a sequence comprising a SNP). In some embodiments, the sequence identity may be sufficient such that the target-specific primer is able to bind to the target sequence and allow for extension by the polymerase, for example the DNA polymerase, as part of the methods as provided herein. In some embodiments, the sequence identity is sufficient such that the target-specific primer is able to bind to the target sequence preferentially to the other target-specific primers which are present in the composition and/or kit.

The term “reaction mixture” refers to an aqueous solution comprising the various reagents for any particular reaction. In the context of the present disclosure relating to a one-step RT PCR method which enables the detection of one or more target sequences, the reaction mixture is capable of first reverse transcribing a RNA (e.g. a target RNA), and then amplifying the reverse transcribed RNA. A reaction mixture typically includes enzymes (e.g. a reverse transcriptase (RT)), buffers, oligonucleotide primers, target nucleic acid, and nucleoside triphosphates. Depending upon the context, the mixture can be either a complete or incomplete reverse transcription reaction mixture.

The term “reverse transcriptase” describes a class of polymerases characterized as RNA-dependent DNA polymerases. Reverse transcriptases typically require a primer to synthesize a DNA transcript from an RNA template. In this manner, reverse transcriptases have been used primarily to transcribe RNA into cDNA, with the subsequent RNA/cDNA duplex being capable of being subjected to further techniques such as PCR for amplifying a target sequence.

The term “buffer,” as used herein, refers to a solution containing a buffering agent or a mixture of buffering agents and further components (e.g. other salts, compounds, enzymes etc.). The nature of the buffer can be critical for enzymatic reactions because they can affect e.g. the stability of the enzymes, but can also provide critical cofactors (e.g. divalent cations) which are necessary for enzymatic activity.

One issue facing one-step RT PCR reactions is that the method requires the enzymatic activity of two enzymes—the reverse transcriptase and the DNA polymerase, and the buffer must therefore be suitable such that both reactions can proceed with efficiency and accuracy.

The present inventors have found two particular aspects of the buffer which is critical for the one-step RT PCR reactions described herein.

Firstly, the inventors have found that the potassium chloride (KCl) concentration cannot be higher than a certain threshold. As shown in the examples herein, increasing the KCl concentration beyond a certain threshold can inhibit the subsequent amplification reaction to the point that the resultant signal is severely diminished.

Therefore, in one embodiment, the buffer comprises KCl and the KCl is at a working concentration of no more than 60 mM, optionally wherein the working concentration of KCl is about 40 mM to about 60 mM, about 45 mM to about 55 mM, or about 50 mM.

The inventors also found that the presence of a non-ionic surfactant is important for the amplification reaction, despite non-ionic surfactants being typically not included in reverse transcriptase buffers.

As used herein, non-ionic surfactants are surfactants comprising a polar, but uncharged hydrophilic group. Examples of non-ionic surfactants are well known in the art and include Cetomacrogol 1000, Cetostearyl alcohol, Cetyl alcohol, Cocamide DEA, Cocamide MEA, Decyl glucoside, IGEPAL (e.g. IGEPAL CA-630), Isoceteth-20, Lauryl glucoside, Monolaurin, Narrow range ethoxylate, Nonidet P-40, Nonoxynol-9, Nonoxynols, NP-40. Octaethylene glycol monododecyl ether, N-Octyl beta-D-thioglucopyranoside, Octyl glucoside, Oleyl alcohol, Pentaethylene glycol monododecyl ether, Poloxamer, Poloxamer 407, Polyglycerol polyricinoleate, Polysorbate, Polysorbate 20, Polysorbate 80 (Polyoxyethylene (20) sorbitan monooleate), Sorbitan monostearate, Sorbitan tristearate, Stearyl alcohol, Tergitol (such as Tergitol 15-S-9, Tergitol 15-S-40, Tergitol 15-S-3), Tween 80, Triton X-114, Tween 20 (Polyoxyethylene (20) sorbitan monolaurate) and Triton X-100.

In one embodiment according to the present invention, the non-ionic surfactant is selected from the group consisting of Tergitol™, Triton, or Igepal. Even more preferably, the non-ionic surfactant is selected from Tergitol 15-S-9, Tergitol 15-S-40, Tergitol 15-S-3, Triton X-100 or Igepal. In one embodiment, the non-ionic surfactant is Tergitol 15-S-40.

A buffer of the present invention may include one or more of the following components, in addition to the KCl and non-ionic surfactant as discussed above.
i) Tris-HCl, preferably Tris-HCl at pH8.3;
ii) MgCl;
iii) Glycerol;
iv) TMAC; and/or
v) an RNase inhibitor.

Enhancer primers as used herein refer to sequence-specific primers which are specific for the same locus as a probe as used herein. Thus, in use, the enhancer primer may be able to compete with the binding of the probe in order to facilitate the amplification reaction and thus generate further tagged/tailed strands for probe binding and signal generation. Thus, in embodiments, the one or more enhancer oligonucleotide primers comprising a sequence having at least 50% identity to the sequence of a probe. In some embodiments, the sequence of the oligonucleotide enhancer primers as used herein can be designed such they are at least 60%, 70%, 80%, 85%, 90%, 95%, 96%, 97%, 98%, 99% or 100% identity to their respective probe/tag sequences. In some embodiments, the sequence identity may be sufficient such that the enhancer primer is able to bind to the tag sequence and allow for extension by the polymerase, for example the DNA polymerase, as part of the methods as provided herein.

In some embodiments, the one or more enhancer oligonucleotide primers may comprise at least two distinct regions, each region comprising a sequence having at least 50% identity to that of the one or more probes. In such embodiments, the two distinct regions may be separated by a linker region. The linker region may be 1 to 10 or 2 to 5 oligonucleotides in length. In some embodiments, there may be no linker region.

For the purpose of this invention, in order to determine the percent identity of two sequences (such as two polynucleotide or two polypeptide sequences), the sequences may be aligned for optimal comparison purposes (e.g., gaps can be introduced in a first sequence for optimal alignment with a second sequence). The nucleotide residues at nucleotide positions may then be compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The terms “complementarity” and “complementary” are interchangeable and refer to the ability of polynucleotides to form base pairs with one another. Base pairs are typically formed by hydrogen bonds between nucleotide units in antiparallel polynucleotide strands or regions.

Complementary polynucleotide strands or regions can base pair in the Watson-Crick manner (e.g., A to T, A to U, C to G). 100% complementarity refers to the situation in which each nucleotide unit of one polynucleotide strand or region can hydrogen bond with each nucleotide unit of a second polynucleotide strand or region of equal length.

The term “probe” as used herein refers to synthetic or biologically produced nucleic acids (DNA or RNA) which, by design or selection, contain specific nucleotide sequences that allow them to hybridize, under defined stringencies, specifically (i.e., preferentially) to target nucleic acid sequences. Thus, probes may also be used interchangeably with “oligonucleotide probe”. In the present teachings, probes may be labelled, e.g. with a reporter label such as a fluorophore or a quencher label. In some embodiments, probes may also comprise at least a pair of labels, comprising a reporter label such as a fluorophore and a quencher label, whereby the quencher label is able to attenuate the emission of the reporter label. The reporter label may be located at a different portion of the probe compared to the quencher label. In some embodiments, the position of the labels is such that when free in solution, the reporter label and quencher label come into close proximity to each other such that little or no emission can be detected from the reporter label.

The term “reporter label” as used herein should be understood to mean a molecule that is capable of emitting a detectable signal and is capable of being quenched by the quencher molecule. Examples of reporter molecules include molecules that are detectable by spectroscopic, photochemical, biochemical, immunochemical, or chemical means. Examples of reporter labels that may be employed include enzymes, enzyme substrates, radioactive atoms, fluorescent dyes, chromophores, chemiluminescent labels, or ligands having specific binding partners.

In one embodiment, the reporter label may be detectable by spectroscopy, and can be a fluorescent dye. The fluorophores for the labelled oligonucleotide pairs may be selected so as to be from a similar chemical family or a different one, such as cyanine dyes, xanthenes or the like. Fluorophores of interest include, but are not limited to fluorescein dyes (e.g. 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′7”-dimethoxy-4′,5′dichloro-6-carboxyfluorescein (JOE)), cyanine dyes such as Cy5, dansyl derivatives, rhodamine dyes (e.g. tetramethyl-6-carboxyrhodamine (TAMRA), and tetrapropano-6-carboxyrhodamine (ROX)), DABSYL, DABCYL, cyanine, such as Cy3, anthraquinone, nitrothiazole, and nitroimidazole compounds, or other non-intercalating dyes. The term “quencher label” should be understood to a “quenching group”, i.e. any fluorescence-modifying chemical group that can attenuate at least partly the light emitted by a fluorescent group. We refer herein to this attenuation as “quenching”. Hence, illumination of the fluorescent group in the close proximity of a quenching group can lead to an emission signal that is less intense than expected, or even completely absent. Quenching occurs through energy transfer between the fluorescent group and the quenching group.

In embodiments of the present invention where the probe comprises both a reporter label such as a fluorophore and a quencher label, the labels are generally positioned such that when free in solution, the reporter label and quencher label come into close proximity to each other such that little or no emission can be detected from the reporter label. This may be, for example, due to the formation of secondary structure due to the sequence of the oligonucleotide probe. In such embodiments, binding of the probes to their respective tag sequences can separate the reporter label and the quencher label such that the level of attenuation from the quencher label is decreased when compared to the probe being free in solution. In other words, binding of the probe to the tag sequence should increase emission from the reporter label on the probe. In one example, this can be by positioning the reporter label and quencher label at opposing ends or end portions of a probe.

The terms “polymerase”, “nucleic acid polymerase” and are used herein in a broad sense and refers to any polypeptide that can catalyse the 5′-to-3′ extension of a hybridized primer by the addition of nucleotides and/or certain nucleotide analogs in a template-dependent manner. In one embodiment, the polymerase is a DNA polymerase. Non-limiting examples of DNA polymerases include RNA-dependent DNA polymerases, including without limitation, reverse transcriptases, and DNA-dependent DNA polymerases. It is to be appreciated that certain DNA polymerases (for example, but not limited to certain eubacterial Type A DNA polymerases and Taq DNA polymerase) may further comprise a structure-specific nuclease activity and that when an amplification reaction comprises an invasive cleavage reaction.

In some embodiments, the polymerase may be a modified polymerase. Polymerases can be modified in their structure either through genetic engineering (i.e. through known molecular biological techniques) or through chemical modification in order to confer certain properties. In other words, a modified polymerase as used herein refers to a polymerase, for example a DNA polymerase which has one or more of: a different primary structure (i.e. amino acid sequence), a different secondary structure, a different tertiary structure or a different quaternary structure to the one or more polymerase/s or fragments thereof, from which it is derived. As referred to above the term modified polymerase also includes within its scope fragments, derivatives and homologues of a modified polymerase as herein defined so long as it retains its polymerase function and modified properties as defined herein.

In some embodiments, the polymerase used may be a polymerase modified to attenuate or completely remove its 5′-3′ exonuclease activity under the conditions used for the polymerisation reaction.

In some embodiments, the polymerase used may be a polymerase modified to be a “hot-start” polymerase. Hot-start polymerases are well known in the art as polymerases which have been modified such that they are completely inactivated at lower temperatures. During a PCR reaction when the temperature is generally increased to >90° C., the modification is reversed and the polymerase becomes active again. This technique allows to reduce non-specific amplification, particularly before the start of a PCR reaction.

Comprising in the context of the present specification is intended to meaning including.

Where technically appropriate, embodiments of the invention may be combined. Embodiments are described herein as comprising certain features/elements. The disclosure also extends to separate embodiments consisting or consisting essentially of said features/elements.

The invention will now be described with reference to the following examples, which are merely illustrative and should not in any way be construed as limiting the scope of the present invention.

### EXAMPLES

### Abbreviations:

FAM: 6-carboxy Fluorescein
HEX: 2′,4′, 5′,7′, 1,4-Hexachlorofluorescein
IABkFQ: Iowa Black® FQ
ROX: 5,6-carboxy-X-rhodamine, SE

### Example 1: Use of an Optimised RT PCR Buffer Compared to Available Reverse Transcriptase Buffers, in a Universal One-Step RT PCR Fluorescent Detection System

There are many existing RT (reverse transcriptase) buffer formulations, as well as optimised buffers available from RT manufacturers. Such formulations differ significantly from those which are described herein. The following experiment was carried out to determine whether known RT buffer formulations can be used effectively in conjunction with the universal fluorescent reporting system in a one-step-RT PCR application in accordance with the methods disclosed herein.

One-step RT PCR was performed using three buffer formulations:
1. Optimised one-step RT PCR buffer in accordance with the present disclosure, for universal fluorescent detection system as described herein.
2. Reverse Transcriptase buffer formulated according to literature searches (e.g. as described in US 2016/0160194 A1).
3. Commercially available RT buffer supplied with commercial Reverse Transcriptase enzyme.

### Buffer Composition 1 at 2×:

1. 10 mM Tris-HCl pH 8.3
2. 100 mM KCl
3. 4.4 mM magnesium chloride
4. 0.05% Tergitol 15-S-40
5. 10% glycerol
6. 20 mM TMAC

### Buffer Composition 2 at 2×:

1. 100 mM Tris-HCl pH8.3
2. 150 mM KCl
3. 6 mM magnesium chloride
4. 10 mM DTT

### Buffer Composition 3 at 2×.

Unknown. The formulation of buffer composition 3 is proprietary to the manufacturer of the RT enzyme but it is the reaction buffer supplied with the Reverse Transcriptase enzyme used in this study.

It should be noted that as the buffer compositions above (and in the examples below) are provided in a “2× composition”, the final working concentrations of each component in the reaction mixture will be half the amount specified. (For example, for buffer 1 above, the final working concentration of KCl will be 50 mM.)

Qualitative endpoint fluorescent detection was performed using human RNA samples.

Target sequence for detection (rs9472138)
TGTCTAGCTAGCTAGCCTTCCATCAGCCATCTTCTTTTTTCATTGTCAG
TGCCTCTTTATGAAGGTAAAATTGACATACCAGAACATATCCTCATTTT
AGGCACACATTTCATGATTTAAGTAAATTTATACAGTTGTGCAACCATC
AGCATAATCTAGTTTTAGAACACTTCTGTTACCCTAAGCACGTTCTCCT
CATA[C/T]CGTTTGTCGTCAATCCCTACCACGGCTACCAGTCTCAGGC
AGCTACTAATCTATCTGCTTTTTTTCTGTGTAATTTTGCCTTTTCCAGA
AAGTCTTTATAGATAGAAGTATACAATGTATAGTCTCTTCTGTTTGGCT
TCTTTCATTAGAATGGTGCTTTTGAGATTCATCTATGTTGTGGCATGTA
TCAGTAGGTTGTT
Allele-specific primer 1:
5′ AGCTGCTATTGTTACCAGTGACGCAGCTCCCTAAGCACGTTCTCCT
CATAC 3′
Allele-specific primer 2:
5′ TGGCCAGTGTATTCTGCACAGGTGGCCACCCTAAGCACGTTCTCCT
CATAT 3′
FAM probe:
/56-FAM/AGCTGCTATTGTTACCAGTGACGCAGCT/3IABKFQ/
HEX probe:
/5HEX/TGGCCAGTGTATTCTGCACAGGTGGCCA/3IABKFQ/
Reverse primer:
5′ GACTGGTAGCCGTGGTAGGGAT 3′

2× one-step RT PCR universal reporting master mixes were prepared using the three buffers described above. The following components were added to each buffer:
1. 0.33 μM allele-specific primer 1
2. 0.33 μM allele-specific primer 2
3. 0.83 μM reverse primer
4. 600 nM FAM probe
5. 600 nM HEX probe
6. 300 μM dNTPs
7. 300 nM 5,6-carboxy-X-rhodamine, SE (5,6-ROX, SE, mixed isomer)
8. 50-100 Units/mL N-terminal truncated Taq Polymerase
9. 2000-10,000 Units/mL modified thermostable Reverse Transcriptase with RNase Inhibitor

A one-step process of reverse transcription and allele-specific PCR amplification was performed in a clear 384-well PCR plate using a 4 μL final reaction volume. To the wells of the PCR plate, 2 μL of 2× genotyping master mix (described above) was added. Addition of PCR master mix was followed by the addition of RNA samples from various individuals at final concentration of 1-10000 copies/μL. The PCR plate was sealed using Star Seal Advanced polyolefin film and thermally cycled on a Peltier-based thermal cycler (MJ PTC-200) using the following thermal cycling conditions:

| | | | | | | # |
|---|---|---|---|---|---|---|
| Step | Description | Temperature | | Time | | Cycles |
| 1 | Reverse | 50° | C. | 10 | min | 1 |
| | Transcription | | | | | |
| 2 | Enzyme | 94° | C. | 15 | min | 1 |
| | activation | | | | | |
| 3 | Template | 94° | C. | 20 | secs | 10 |
| | denaturation | | | | | |
| | Annealing and | 65-57° | C. | 60 secs (drop | | |
| | extension | | | 0.8° C. per | | |
| | | | | cycle) | | |
| 4 | Denaturation | 94° | C. | 20 | secs | 30 |
| | Annealing and | 57° | C. | 60 | secs | |
| | extension | | | | | |

After thermal-cycling, endpoint fluorescence was recorded using a Tecan Spark fluorescent plate reader with the monochromator set to the following wavelengths:
FAM excitation: 485 nm, FAM Emission: 520 nm
HEX excitation: 535 nm, HEX Emission: 556 nm
ROX excitation: 575 nm, ROX Emission: 610 nm

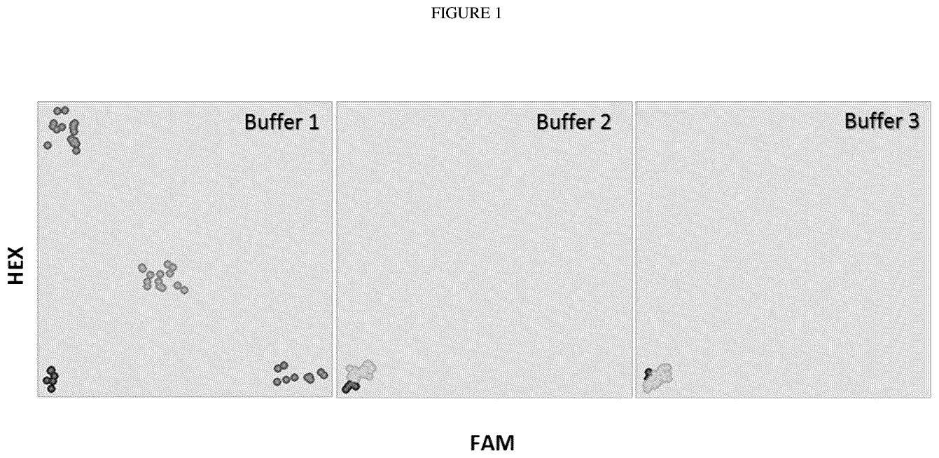

The data obtained was then plotted as FAM signal divided by ROX on the X axis, and HEX signal divided by ROX on the Y axis (FIG. 1).

### Conclusions

As can be seen above, well-defined endpoint genotyping clusters were obtained with the optimised buffer formulation disclosed herein (Buffer 1), showing that both reverse transcription and the subsequent PCR step occurred successfully in a one-step reaction. However, when the universal one-step RT PCR fluorescent detection system was used with either a reaction buffer described in the literature (Buffer 2) or the reaction buffer supplied by the reverse transcriptase manufacturer (Buffer 3), it was observed that the samples failed to amplify.

The conclusion is that the existing buffer formulations for RT enzymes do not function as part of the one-step RT fluorescent detection system described herein. However, the optimised buffer formulation (Buffer 1) enables the system to work as a one-step system. The commercially available or search-derived formulations will work well for the process of reverse transcription as an isolated step, but they do not permit the subsequent PCR step in the same reaction.

### Example 2: One-Step RT-PCR Method for Qualitative Endpoint Detection in Conjunction with Universal Fluorescent Reporting System

Sequence (SARS-CoV-2 mutation N501Y)
AAGGTTTTAATTGTTACTTTCCTTTACAATCATATGGTTTCCAACCCAC
T[A/T]ATGGTGTTGGTTACCAACCATACAGAGTAGTAGTACTTTCTTT
TGAACTT
Allele-specific primer 1:
5′AGCTGCTATTGTTACCAGTGACGCAGCTACAATCATATGGTTTCCAA
CCCACTA 3′
Allele-specific primer 2:
5′TGGCCAGTGTATTCTGCACAGGTGGCCAACAATCATATGGTTTCCAA
CCCACTT 3′
Reverse primer:
5′ CTCTGTATGGTTGGTAACCAACACCAT 3′
FAM probe:
/56-FAM/AGCTGCTATTGTTACCAGTGACGCAGCT/3IABKFQ/
HEX probe:
/5HEX/TGGCCAGTGTATTCTGCACAGGTGGCCA/3IABKFQ/
Enhancer primer A1:
5′ GCTGCTATTGTTACCAGTGACGCAGCT 3′
Enhancer primer A2:
5′ GGCCAGTGTATTCTGCACAGGTGGCCA 3′

All oligonucleotides were hydrated to 100 μM (initial concentration) using Te buffer (10 mM Tris-HCl pH 8.3 with 0.1 mM EDTA).

A 2× genotyping master mix including assay primers was created which included the following components:
1. 0.33 μM allele-specific primer 1
2. 0.33 μM allele-specific primer 2
3. 0.83 μM reverse primer
4. 600 nM FAM probe
5. 600 nM HEX probe
6. 10 mM Tris-HCl pH 8.3
7. 100 mM KCl
8. 4.4 mM magnesium chloride
9. 300 μM dNTPs
10. 300 nM 5,6-carboxy-X-rhodamine, SE (5,6-ROX, SE, mixed isomer)
11. 0.05% Tergitol 15-S-40
12. 10% glycerol
13. 20 mM TMAC
14. 50-100 Units/mL N-terminal truncated Taq Polymerase
15. 2000-10,000 Units/mL modified thermostable Reverse Transcriptase with RNase Inhibitor
16. 80 nM enhancer primer A1
17. 80 nM enhancer primer A2

A one-step process of reverse transcription and allele-specific PCR amplification was performed on a clear 384-well PCR plate using 4 μL final reaction volume. To the wells of the PCR plate, 2 μL of 2× genotyping master mix (described above) was added. Addition of PCR master mix was followed by the addition of RNA samples from various individuals at final concentration of 1-10000 copies/μL. The PCR plate was sealed using Star Seal Advanced polyolefin film. The plate was then thermal cycled on Peltier thermal cycler MJ PTC-200 using the following thermal cycling conditions:

| | | | | | | # |
|---|---|---|---|---|---|---|
| Step | Description | Temperature | | Time | | Cycles |
| 1 | Reverse | 50° | C. | 10 | min | 1 |
| | Transcription | | | | | |
| 2 | Enzyme | 94° | C. | 15 | min | 1 |
| | activation | | | | | |
| 3 | Template | 94° | C. | 20 | secs | 10 |
| | denaturation | | | | | |
| | Annealing and | 65-57° | C. | 60 secs (drop | | |
| | extension | | | 0.8° C. per | | |
| | | | | cycle) | | |
| 4 | Denaturation | 94° | C. | 20 | secs | 30 |
| Annealing and | 57° C. | 60 | secs | | | |
| extension | | | | | | |

After thermal-cycling, endpoint fluorescence was recorded using a Tecan Spark fluorescent plate reader with the monochromator set to the following wavelengths:
FAM excitation: 485 nm, FAM emission: 520 nm
HEX excitation: 535 nm, HEX emission: 556 nm
ROX excitation: 575 nm, ROX emission: 610 nm

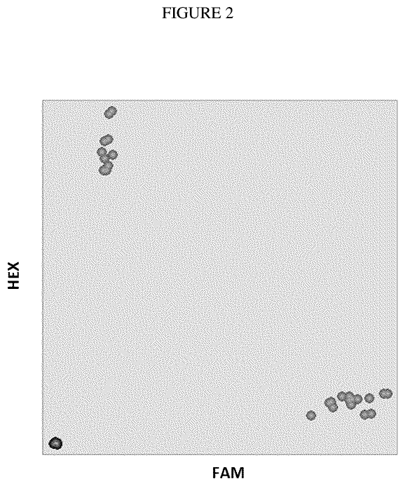

The data obtained was then plotted as FAM signal divided by ROX on the x-axis, and HEX signal divided by ROX on the y-axis (FIG. 2).

### Conclusions

The one-step RT PCR universal reporting method was observed to correctly genotype the N501Y mutation in SARS-CoV-2. Two well-defined genotyping clusters were observed where all the individuals with the wild-type allele (A) reported with FAM and individuals with the mutant allele (T) reported with HEX. Use of the technology in this manner demonstrates its applicability to the field of high-throughput diagnostics, to which it is also relevant due to its much lower cost compared to hydrolysis probe-based approaches.

### Example 3: Quantitative Real-Time Sequence Detection of PCR Product from RNA Samples with One-Step RT PCR Universal Fluorescent Reporting System

One-step RT PCR was performed on a serial dilution of RNA samples using the one-step RT qPCR universal reporting method described herein.

Sequence
TCAGGACTTGTTCTTACCTTTCTTTTCCAATGTTACTTGGTTCCATGCT
ATACATGTCTCTGGGACCAATGGTACTAAGAGGTTTGATAACCCTGTCC
TAC
Forward primer:
5′ GAAGGTGACCAAGTTCATGCTTAGTACCATTGGTCCCAGAGAC 3′
Reverse primer:
5′ CCTTTCTTTTCCAATGTTACTTGGTTCCAT 3′
FAM probe:
/56-FAM/GAAGGTGACCAAGTTCATGCT/3IABKFQ/
Enhancer primer:
5′ GAAGGTGACCAAGTTCATGCT 3′

All oligonucleotides were hydrated to 100 μM (initial concentration) using Te buffer (10 mM Tris-HCl pH 8.3 with 0.1 mM EDTA).

A 2× One step RT PCR master mix including assay primers was created which included the following components:
1. 0.33 μM forward primer
2. 0.83 μM reverse primer
3. 600 nM FAM probe
4. 10 mM Tris-HCl pH 8.3
5. 100 mM KCl
6. 4.4 mM magnesium chloride
7. 300 μM dNTPs
8. 0.05% Tergitol 15-S-40
9. 10% glycerol
10. 20 mM TMAC
11. 50-100 Units/mL N-terminal truncated Taq Polymerase
12. 2000-10,000 Units/mL modified thermostable Reverse Transcriptase with RNase Inhibitor

One-step process of reverse transcription and allele specific PCR amplification assay was performed on a clear 384-well PCR plate using 10 μL final reaction volume. To the wells of the PCR plate, 5 μL of 2× one-step RT PCR master mix (described above) was added. Addition of PCR master mix was followed by a 1/10 serial dilution addition of RNA samples with a final concentration of 1-10000 copies/μL. The PCR plate was sealed using Star Seal Advanced polyolefin film. The plate was then thermal cycled on an ABI 7900 qPCR instrument using the following thermal cycling conditions:

| | | | | | | # |
|---|---|---|---|---|---|---|
| Step | Description | Temperature | | Time | | Cycles |
| 1 | Reverse | 50° | C. | 10 | min | 1 |
| | Transcription | | | | | |
| 2 | Enzyme | 94° | C. | 15 | min | 1 |
| | activation | | | | | |
| 3 | Template | 94° | C. | 20 | secs | 10 |
| | denaturation | | | | | |
| | Annealing and | 65-57° | C. | 60 secs (drop | | |
| | extension | | | 0.8° C. per | | |
| | | | | cycle) | | |
| 4 | Denaturation | 94° | C. | 20 | secs | 30 |
| | Annealing and | 57° | C. | 60 | secs | |
| | extension | | | | | |

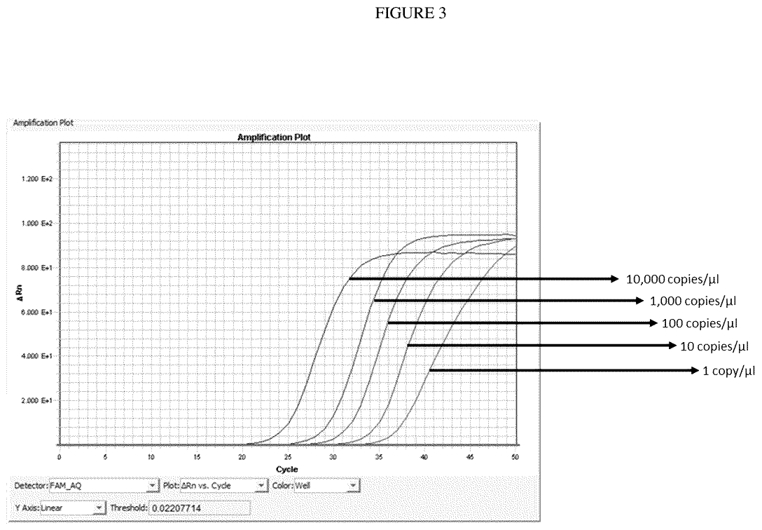

Fluorescence was recorded in real-time at each PCR cycle during the annealing and extension step. The resulting data was plotted as delta Rn vs cycle number and Ct values were calculated automatically using ABI 7900 software (FIG. 3).

### Conclusion

The one-step RT qPCR universal reporting method was observed to function correctly in qPCR/real-time PCR mode. Analysis was possible over a very large dynamic range and demonstrated very high sensitivity, despite literature claims that one-step systems are inferior to two-step systems in this regard.

### Example 4: To Study the Effect of Varying Concentrations of Potassium Chloride in the One-Step RT PCR Buffer for Universal Fluorescent Detection System

One step RT PCR was performed using varying concentrations of potassium chloride. Qualitative endpoint fluorescent detection was performed using human RNA samples.

### Buffer Composition at 2×:

1. 10 mM Tris-HCl pH 8.3
2. 100 mM KCl/120 mM KCl/140 mM KCl
3. 4.4 mM magnesium chloride
4. 0.05% Tergitol 15-S-40
5. 10% glycerol
6. 20 mM TMAC

Target sequence (rs10506440)
AAATTAAATGTTACTACATTTGTAACCATGAAGTAAATCAGCCTAAGAT
GTTTAAAACAAATCTCAGTAGTTTAACATATTGGTTTTACAAAACAAAT
TACATTTATTAGCAGAAGGAGAGGTACAGGCTGCTACCTGTTTAAAAAA
ATTGTAACAAGTATGATTAAGAAAATATATATGACAAAGTATCATAAAA
ATGT[G/T]TGTTGGATTAGATAGTAAATACTAGATGAACTGCATTTGA
ATTTTTTCAATGCAGTGAAAGACAAGTTTGTAAAGTCCTCTTTTGAATT
GATGTAAGCTTGTGAGTATATCCCAACGGTCATTTGGAGCCTCCCTTTT
GCCATATTTGTCTAATGGATCACATATCAGGCCTATTACTATTTTCAGA
AACAGTATTCATG
Allele-specific primer 1:
5′AGCTGCTATTGTTACCAGTGACGCAGCTCATCTAGTATTTACTATCT
AATCCAACAC 3′
Allele-specific primer 2:
5′TGGCCAGTGTATTCTGCACAGGTGGCCACATCTAGTATTTACTATCT
AATCCAACAA 3′
Reverse primer:
5′ CAGAAGGAGAGGTACAGGCTGCTA 3′
FAM probe:
/56-FAM/AGCTGCTATTGTTACCAGTGACGCAGCT/3IABKFQ/
HEX probe:
/5HEX/TGGCCAGTGTATTCTGCACAGGTGGCCA/3IABKFQ/

All oligonucleotides were hydrated to 100 μM (initial concentration) using Te buffer (10 mM Tris-HCl pH 8.3 with 0.1 mM EDTA).

A 2× RT PCR master mix was prepared using each of the three KCl concentrations described above. The following additional components were added to the buffer:
1. 0.33 μM allele-specific primer 1
2. 0.33 μM allele-specific primer 2
3. 0.83 μM reverse primer 3
4. 600 nM FAM probe
5. 600 nM HEX probe
6. 300 μM dNTPs
7. 300 nM 5,6-carboxy-X-rhodamine, SE (5,6-ROX, SE, mixed isomer)
8. 50-100 Units/mL N-terminal truncated Taq Polymerase
9. 2000-10,000 Units/mL modified thermostable Reverse Transcriptase with RNase Inhibitor A one-step process of reverse transcription and allele-specific PCR amplification was performed on a clear 384-well PCR plate using 4 μL final reaction volume. To the wells of the PCR plate, 2 μL of 2× genotyping master mix (described above) was added. Addition of PCR master mix was followed by the addition of RNA sample from various individuals at final concentration of 1-10000 copies/μL. The PCR plate was sealed using Star Seal Advanced Polyolefin film. The plate was then thermally cycled on a Peltier thermal cycler (MJ PTC-200) using the following thermal cycling conditions:

| | | | | | | # |
|---|---|---|---|---|---|---|
| Step | Description | Temperature | | Time | | Cycles |
| 1 | Reverse | 50° | C. | 10 | min | 1 |
| | Transcription | | | | | |
| 2 | Enzyme | 94° | C. | 15 | min | 1 |
| | activation | | | | | |
| 3 | Template | 94° | C. | 20 | secs | 10 |
| | denaturation | | | | | |
| | Annealing and | 65-57° | C. | 60 secs (drop | | |
| | extension | | | 0.8° C. per | | |
| | | | | cycle) | | |
| 4 | Denaturation | 94° | C. | 20 | secs | 30 |
| | Annealing and | 57° | C. | 60 | secs | |
| | extension | | | | | |

After thermal-cycling, endpoint fluorescence was recorded using a Tecan Spark fluorescent plate reader with the monochromator set to the following wavelengths:
FAM excitation: 485 nm, FAM emission: 520 nm
HEX excitation: 535 nm, HEX emission: 556 nm
ROX excitation: 575 nm, ROX emission: 610 nm

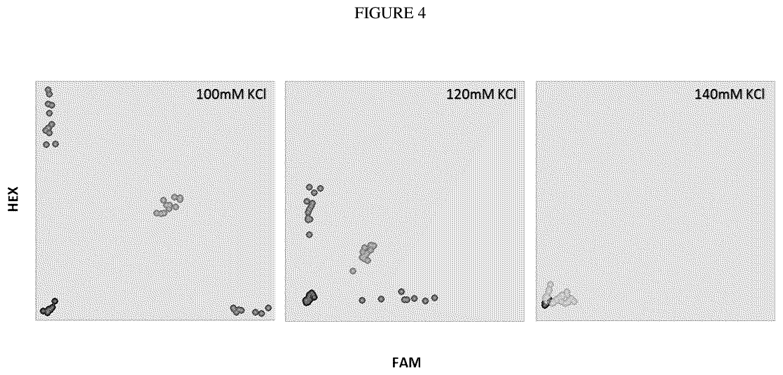

The data obtained was then plotted as FAM signal divided by ROX on the x-axis, and HEX signal divided by ROX on the y-axis (FIG. 4).

### Conclusions

Very significantly reduced amplification and decreased fluorescence were observed with increasing concentrations of KCl in the buffer system. Additional thermal cycles did not lead to significant improvement in the data. A concentration of 150 mM KCl is generally described as required in commercially available buffer compositions (at 2× concentration) for reverse transcriptase activity (see Buffer 2 as tested in Example 1). However, this concentration of KCl clearly inhibits PCR amplification, thus the present disclosure provides an improved buffer formulation which is able to facilitate PCR amplification without reverse transcriptase inhibition.

### Example 5: To Assess the Effect of Detergent/Surfactant in One-Step Universal Fluorescent Detection System

Manufacturers of commercially available RT buffers do not list a detergent or surfactant amongst the required components. The purpose of this experiment was to demonstrate that a one-step system required the presence of a detergent or surfactant to be present in the buffer formulation for the one-step RT genotyping method to function correctly.

Target sequence (rs4073)
GTTAACATGCATTAGAAAACTAAATCTTTTGAAAAGTTGTAGTATGCCC
CCTAAGAGCAGTAACAGTTCCTAGAAACTCTCTAAAATGCTTAGAAAAA
GATTTATTTTAAATTACCTCCCCAATAAAATGATTGGCTGGCTTATCTT
CACCATCATGATAGCATCTGTAATTAACTGAAAAAAAATAATTATGCCA
TTAAAAGAAAATCATCCATGATCTTGTTCTAACACCTGCCACTCTAGTA
CTATATCTGTCACATGGTACTATGATAAAGTTATCTAGAAATAAAAAAG
CATACA[A/T]TTGATAATTCACCAAATTGTGGAGCTTCAGTATTTTAA
ATGTATATTAAAATTAAATTATTTTAAAGATCAAAGAAAACTTTCGTCA
TACTCCGTATTTGATAAGGAACAAATAGGAAGTGTGATGACTCAGGTTT
GCCCTGAGGGGATGGGCCATCAGTTGCAAATCGTGGAATTTCCTCTGAC
ATAATGAAAAGATGAGGGTGCATAAGTTCTCTAGTAGGGTGATGATATA
AAAAGCCACCGGAGCACTCCATAAGGCACAAACTTTCAGAGACAGCAGA
GCACACAAGCTTCTAGG
Allele-specific primer 1:
5′AGCTGCTATTGTTACCAGTGACGCAGCTGAAGCTCCACAATTTGGT
GAATTATCAAT 3′
Allele-specific primer 2:
5′TGGCCAGTGTATTCTGCACAGGTGGCCAGAAGCTCCACAATTTGGT
GAATTATCAAA 3′
Reverse primer:
5′ CACTCTAGTACTATATCTGTCACATGGTA 3′
FAM probe:
/56-FAM/AGCTGCTATTGTTACCAGTGACGCAGCT/3IABKFQ/
HEX probe:
/5HEX/TGGCCAGTGTATTCTGCACAGGTGGCCA/3IABKFQ/
Enhancer primer A1:
5′ GCTGCTATTGTTACCAGTGACGCAGCT 3′
Enhancer primer A2:
5′ GGCCAGTGTATTCTGCACAGGTGGCCA 3′

All oligonucleotides were hydrated to 100 μM (initial concentration) using Te buffer (10 mM Tris-HCl pH 8.3 with 0.1 mM EDTA).

A 2× genotyping master mix including assay primers was created which included the following components:
1. 0.33 μM allele-specific primer 1
2. 0.33 μM allele-specific primer 2
3. 0.83 μM reverse primer
4. 600 nM FAM probe
5. 600 nM HEX probe
6. 10 mM Tris-HCl pH 8.3
7. 100 mM KCl
8. 4.4 mM magnesium chloride
9. 300 μM dNTPs
10. 300 nM 5,6-carboxy-X-rhodamine, SE (5,6-ROX, SE, mixed isomer)
11. 0.05% Tergitol 15-S-40 or none
12. 10% Glycerol
13. 20 mM TMAC
14. 50-100 Units/mL N-terminal truncated Taq Polymerase
15. 2000-10,000 Units/mL modified thermostable Reverse Transcriptase with RNase Inhibitor
16. 80 nM enhancer primer A1
17. 80 nM enhancer primer A2

A one-step process of reverse transcription and allele-specific PCR amplification assay was performed on a clear 384-well PCR plate using 4 μL final reaction volume. To the wells of the PCR plate, 2 μL of 2× genotyping master mix (described above) was added. Addition of PCR master mix was followed by the addition of RNA sample from various individuals at final concentration of 1-10000 copies/μL. The PCR plate was sealed using Star Seal Advanced Polyolefin film. The plate was then thermally cycled on Peltier thermal cycler (MJ PTC-200) using the following thermal cycling conditions:

| | | | | | | # |
|---|---|---|---|---|---|---|
| Step | Description | Temperature | | Time | | Cycles |
| 1 | Reverse | 50° | C. | 10 | min | 1 |
| | Transcription | | | | | |
| 2 | Enzyme | 94° | C. | 15 | min | 1 |
| | activation | | | | | |
| 3 | Template | 94° | C. | 20 | secs | 10 |
| | denaturation | | | | | |
| | Annealing and | 65-57° | C. | 60 secs (drop | | |
| | extension | | | 0.8° C. per | | |
| | | | | cycle) | | |
| 4 | Denaturation | 94° | C. | 20 | secs | 30 |
| | Annealing and | 57° | C. | 60 | secs | |
| | extension | | | | | |

After thermal-cycling, endpoint fluorescence was recorded using a Tecan Spark fluorescent plate reader with the monochromator set to the following wavelengths:
FAM excitation: 485 nm, FAM emission: 520 nm
HEX excitation: 535 nm, HEX emission: 556 nm
ROX excitation: 575 nm, ROX emission: 610 nm

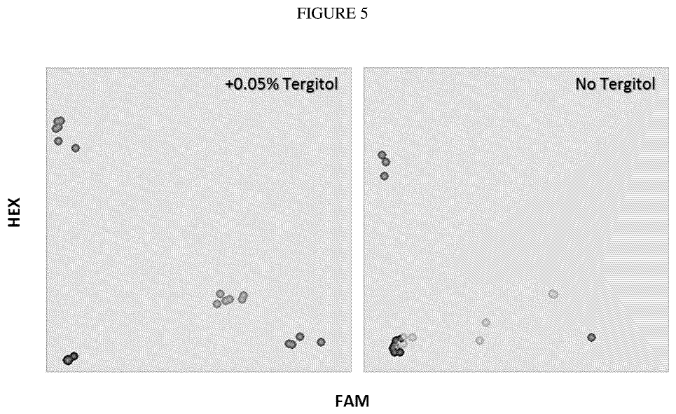

The data obtained was then plotted as FAM signal divided by ROX on the x-axis, and HEX signal divided by ROX on the y-axis (FIG. 5).

### Conclusions

All samples amplified and clustered well in three genotyping groups in the presence of 0.05% Tergitol whereas more than half of the samples failed to amplify at all in the absence of Tergitol. Furthermore, those samples that did amplify in the absence of Tergitol did so poorly in many cases, and did not cluster distinctively to indicate the presence of any given allele. This clearly shows that a surfactant is required for a one-step RT PCR buffer system to function correctly. Additional thermal cycles did not lead to significant improvement in the data.

It will of course be understood that, although the present invention has been described by way of example, the examples are in no way meant to be limiting, and modifications can be made within the scope of the claims hereinafter. Preferred features of each embodiment of the invention are as for each of the other embodiments mutatis mutandis. All publications, including but not limited to patents and patent applications, cited in this specification are herein incorporated by reference as if each individual publication was specifically and individually indicated to be incorporated by reference herein.

1. A method for detecting one or more target sequences in a sample comprising RNA by amplification, the method comprising:
a) providing an aqueous composition comprising:
i) one or more forward oligonucleotide primers having a 5′ region which is a tag sequence and a 3′ region specific for a target sequence;
ii) a reverse oligonucleotide primer, such that a forward and the reverse primer operably form a primer pair;
iii) one or more probes having a reporter label and a quencher label, each probe comprising a sequence having at least 50% identity to the tag sequence of a forward oligonucleotide primer; and
iv) optionally one or more enhancer oligonucleotide primers comprising a sequence having at least 50% identity to the sequence of a probe;
b) combining the primers, the probes, the sample, a polymerase, and a reverse transcriptase in a reaction mixture with a buffer; and
c) performing a one-step polymerase chain reaction (PCR) on the reaction mixture to generate tagged nucleic acids, whereby the probe(s) can bind the tagged nucleic acids.

2. A kit for use in a nucleic acid amplification process, comprising:
i) one or more forward oligonucleotide primers having a 5′ region which is a tag sequence and a 3′ region specific for a target sequence;
ii) a reverse oligonucleotide primer, such that a forward and the reverse primer operably form a primer pair;
iii) one or more probes having a reporter label and a quencher label, each probe comprising a sequence having at least 50% identity to the tag sequence of a forward oligonucleotide primer;
iv) a polymerase;
v) a reverse transcriptase;
vi) a buffer; and
vii) optionally one or more enhancer oligonucleotide primers comprising a sequence having at least 50% identity to the sequence of a probe.

3. The method according to claim 1, wherein the buffer comprises KCl and the KCl is at a working concentration of no more than 60 mM, optionally wherein the working concentration of KCl is about 40 mM to about 60 mM, about 45 mM to about 55 mM, or about 50 mM.

4. The method according to claim 1, wherein the buffer comprises a non-ionic surfactant.

5. The method to claim 4, wherein the non-ionic surfactant is selected from Tergitol™, Triton, or Igepal.

6. The method according to claim 4, wherein the non-ionic surfactant is selected from Tergitol 15-S-9, Tergitol 15-S-40, Tergitol 15-S-3, Triton X-100 or Igepal, optionally wherein the non-ionic surfactant is Tergitol 15-S-40.

7. The method according to claim 4, wherein the non-ionic surfactant is present at a working concentration of at least about 0.01%, optionally wherein the non-ionic surfactant is present at a working concentration of about 0.05%.

8. The method according to claim 1, wherein the buffer further comprises one or more of:
i) Tris-HCl, preferably Tris-HCl at pH 8.3;
ii) MgCl;
iii) Glycerol;
iv) TMAC; and/or
v) an RNase inhibitor.

9. The method according to claim 1, wherein the polymerase is a DNA polymerase, optionally wherein the polymerase is a fragment or domain or a derivative of Taq polymerase.

10. The method according to claim 1, wherein the polymerase is a modified and/or truncated polymerase.

11. The method according to claim 9, wherein the polymerase is modified to remove exo-nuclease activity and/or modified to be a “hot-start” polymerase.

12. The method according to claim 1, wherein the reverse transcriptase is a modified reverse transcriptase, optionally wherein the reverse transcriptase is modified to be thermostable.

13. The method according to claim 1, wherein the one or more enhancer oligonucleotide primers comprise at least two distinct regions, each region comprising a sequence having at least 50% identity to that of the one or more probes.

14. The method according to claim 13, wherein the two distinct regions are separated by a linker region, optionally wherein the linker region is 1 to 10 or 2 to 5 oligonucleotides in length.

15. The method according to claim 1, wherein the one or more probes comprise a sequence having at least 60%, 70%, 80%, 90%, 95%, 98% or 99% identity to the tag sequence of a forward oligonucleotide primer.

16. The method according to claim 1, wherein the one or more probes comprise a sequence identical to the tag sequence of a forward oligonucleotide primer.

17. The method according to claim 1, wherein the one or more enhancer oligonucleotide primers comprise a sequence having at least 60%, 70%, 80%, 90%, 95%, 98% or 99% identity to the sequence of a probe.

18. The method according to claim 1, wherein the one or more enhancer oligonucleotide primers comprise a sequence identical to the sequence of a probe.

19. The method according to claim 1, wherein the one-step polymerase chain reaction (PCR) comprises at least 4, at least 10, at least 20, or at least 30 cycles of PCR.

20. The method according to claim 1, wherein the one-step polymerase chain reaction (PCR) step comprises a reverse transcription step prior to the PCR cycles.

21. The method according to claim 1, further comprising: d) measuring the signal generated by the binding of the probes to the tagged nucleic acids.

22. The method according to claim 21, wherein the measurement is made (i) in real time or (ii) at the end of the reaction.

23. The method according to claim 1, wherein the total volume of the reaction mixture is under 1 μL.

24. A buffer composition suitable for a one-step reverse transcriptase polymerase chain reaction (RT-PCR), wherein the buffer comprises:
(a) KCl and the KCl is at a working concentration of no more than 60 mM, optionally wherein the working concentration of KCl is about 40 mM to about 60 mM, about 45 mM to about 55 mM, or about 50 mM; and
(b) a non-ionic surfactant.

25. The buffer composition according to claim 24, wherein the non-ionic surfactant is selected from Tergitol™, Triton, or Igepal.

26. The buffer composition according to claim 25, wherein the non-ionic surfactant is selected from Tergitol 15-S-9, Tergitol 15-S-40, Tergitol 15-S-3, Triton X-100 or Igepal,
optionally wherein the non-ionic surfactant is Tergitol-S-40.

27. The buffer composition according to claim 24, wherein the non-ionic surfactant is present at a working concentration of at least about 0.01%, optionally wherein the non-ionic surfactant is present at a working concentration of about 0.05%.

28. The kit according to claim 2, wherein the buffer comprises KCl and the KCl is at a working concentration of no more than 60 mM, optionally wherein the working concentration of KCl is about 40 mM to about 60 mM, about 45 mM to about 55 mM, or about 50 mM.

29. The kit according to claim 2, wherein the buffer comprises a non-ionic surfactant.

30. The kit according to claim 29, wherein the non-ionic surfactant is selected from Tergitol™, Triton, or Igepal.

31. The kit according to claim 29, wherein the non-ionic surfactant is selected from Tergitol 15-S-9, Tergitol 15-S-40, Tergitol 15-S-3, Triton X-100 or Igepal, optionally wherein the non-ionic surfactant is Tergitol 15-S-40.

32. The kit according to claim 29, wherein the non-ionic surfactant is present at a working concentration of at least about 0.01%, optionally wherein the non-ionic surfactant is present at a working concentration of about 0.05%.

33. The kit according to claim 2, wherein the buffer further comprises one or more of:
i) Tris-HCl, preferably Tris-HCl at pH8.3;
ii) MgCl;
iii) Glycerol;
iv) TMAC; and/or
v) an RNase inhibitor.

34. The kit according to claim 2, wherein the polymerase is a DNA polymerase, optionally wherein the polymerase is a fragment or domain or a derivative of Taq polymerase.

35. The kit according to claim 2, wherein the polymerase is a modified and/or truncated polymerase.

36. The kit according to claim 34, wherein the polymerase is modified to remove exo-nuclease activity and/or modified to be a “hot-start” polymerase.

37. The kit according to claim 2, wherein the one or more enhancer oligonucleotide primers comprise at least two distinct regions, each region comprising a sequence having at least 50% identity to that of the one or more probes.

38. The kit according to claim 37, wherein the two distinct regions are separated by a linker region, optionally wherein the linker region is 1 to 10 or 2 to 5 oligonucleotides in length.

39. The kit according to claim 2, wherein the one or more probes comprise a sequence having at least 60%, 70%, 80%, 90%, 95%, 98% or 99% identity to the tag sequence of a forward oligonucleotide primer.

40. The kit according to claim 2, wherein the one or more probes comprise a sequence identical to the tag sequence of a forward oligonucleotide primer.

41. The kit according to claim 2, wherein the one or more enhancer oligonucleotide primers comprise a sequence having at least 60%, 70%, 80%, 90%, 95%, 98% or 99% identity to the sequence of a probe.

42. The kit according to claim 2, wherein the one or more enhancer oligonucleotide primers comprise a sequence identical to the sequence of a probe.


SEQUENCE LISTING (33 sequences)

SEQ ID NO: 1 - DNA, 402 bp, Homo sapiens
  source 1..402: mol_type = other DNA
tgtctagctagctagccttccatcagccatcttcttttttcattgtcagtgcctctttatgaaggtaaaattgacataccagaacatatcctcattttaggcacacatttcatgatttaagtaaatttatacagttgtgcaaccatcagcataatctagttttagaacacttctgttaccctaagcacgttctcctcatactcgtttgtcgtcaatccctaccacggctaccagtctcaggcagctactaatctatctgctttttttctgtgtaattttgccttttccagaaagtctttatagatagaagtatacaatgtatagtctcttctgtttggcttctttcattagaatggtgcttttgagattcatctatgttgtggcatgtatcagtaggttgtt

SEQ ID NO: 2 - DNA, 51 bp, synthetic construct
  source 1..51: mol_type = other DNA
agctgctattgttaccagtgacgcagctccctaagcacgttctcctcatac

SEQ ID NO: 3 - DNA, 51 bp, synthetic construct
  source 1..51: mol_type = other DNA
tggccagtgtattctgcacaggtggccaccctaagcacgttctcctcatat

SEQ ID NO: 4 - DNA, 28 bp, synthetic construct
  source 1..28: mol_type = other DNA
  misc_feature 1: note = 5' 6-carboxyfluorescein (6-FAM)
  misc_feature 28: note = 3' Iowa Black FQ (IABkFQ)
agctgctattgttaccagtgacgcagct

SEQ ID NO: 5 - DNA, 28 bp, synthetic construct
  source 1..28: mol_type = other DNA
  misc_feature 28: note = 3' Iowa Black FQ (IABkFQ)
  misc_feature 1: note = 5' HEX (HEX is 2',4', 5',7', 1,4- Hexachlorofluorescein)
tggccagtgtattctgcacaggtggcca

SEQ ID NO: 6 - DNA, 22 bp, synthetic construct
  source 1..22: mol_type = other DNA
gactggtagccgtggtagggat

SEQ ID NO: 7 - DNA, 101 bp, SARS-CoV-2
  source 1..101: mol_type = other DNA
aaggttttaattgttactttcctttacaatcatatggtttccaacccactwatggtgttggttaccaaccatacagagtagtagtactttcttttgaactt

SEQ ID NO: 8 - DNA, 54 bp, synthetic construct
  source 1..54: mol_type = other DNA
agctgctattgttaccagtgacgcagctacaatcatatggtttccaacccacta

SEQ ID NO: 9 - DNA, 54 bp, synthetic construct
  source 1..54: mol_type = other DNA
tggccagtgtattctgcacaggtggccaacaatcatatggtttccaacccactt

SEQ ID NO: 10 - DNA, 27 bp, synthetic construct
  source 1..27: mol_type = other DNA
ctctgtatggttggtaaccaacaccat

SEQ ID NO: 11 - DNA, 28 bp, synthetic construct
  source 1..28: mol_type = other DNA
  misc_feature 1: note = 5, 6-carboxyfluorescein (6-FAM)
  misc_feature 28: note = 3' Iowa Black FQ (IABkFQ)
agctgctattgttaccagtgacgcagct

SEQ ID NO: 12 - DNA, 28 bp, synthetic construct
  source 1..28: mol_type = other DNA
  misc_feature 28: note = 3' Iowa Black FQ (IABkFQ)
  misc_feature 1: note = 5' HEX (HEX is 2',4', 5',7', 1,4- Hexachlorofluorescein)
tggccagtgtattctgcacaggtggcca

SEQ ID NO: 13 - DNA, 27 bp, synthetic construct
  source 1..27: mol_type = other DNA
gctgctattgttaccagtgacgcagct

SEQ ID NO: 14 - DNA, 27 bp, synthetic construct
  source 1..27: mol_type = other DNA
ggccagtgtattctgcacaggtggcca

SEQ ID NO: 15 - DNA, 101 bp, unidentified
  source 1..101: mol_type = other DNA
tcaggacttgttcttacctttcttttccaatgttacttggttccatgctatacatgtctctgggaccaatggtactaagaggtttgataaccctgtcctac

SEQ ID NO: 16 - DNA, 43 bp, synthetic construct
  source 1..43: mol_type = other DNA
gaaggtgaccaagttcatgcttagtaccattggtcccagagac

SEQ ID NO: 17 - DNA, 30 bp, synthetic construct
  source 1..30: mol_type = other DNA
cctttcttttccaatgttacttggttccat

SEQ ID NO: 18 - DNA, 21 bp, synthetic construct
  source 1..21: mol_type = other DNA
  misc_feature 1: note = 5' 6-carboxyfluorescein (6-FAM)
  misc_feature 21: note = 3' Iowa Black FQ (IABkFQ)
gaaggtgaccaagttcatgct

SEQ ID NO: 19 - DNA, 21 bp, synthetic construct
  source 1..21: mol_type = other DNA
gaaggtgaccaagttcatgct

SEQ ID NO: 20 - DNA, 401 bp, Homo sapiens
  source 1..401: mol_type = other DNA
aaattaaatgttactacatttgtaaccatgaagtaaatcagcctaagatgtttaaaacaaatctcagtagtttaacatattggttttacaaaacaaattacatttattagcagaaggagaggtacaggctgctacctgtttaaaaaaattgtaacaagtatgattaagaaaatatatatgacaaagtatcataaaaatgtktgttggattagatagtaaatactagatgaactgcatttgaattttttcaatgcagtgaaagacaagtttgtaaagtcctcttttgaattgatgtaagcttgtgagtatatcccaacggtcatttggagcctcccttttgccatatttgtctaatggatcacatatcaggcctattactattttcagaaacagtattcatg

SEQ ID NO: 21 - DNA, 57 bp, synthetic construct
  source 1..57: mol_type = other DNA
agctgctattgttaccagtgacgcagctcatctagtatttactatctaatccaacac

SEQ ID NO: 22 - DNA, 57 bp, synthetic construct
  source 1..57: mol_type = other DNA
tggccagtgtattctgcacaggtggccacatctagtatttactatctaatccaacaa

SEQ ID NO: 23 - DNA, 24 bp, synthetic construct
  source 1..24: mol_type = other DNA
cagaaggagaggtacaggctgcta

SEQ ID NO: 24 - DNA, 28 bp, synthetic construct
  source 1..28: mol_type = other DNA
  misc_feature 28: note = 3' Iowa Black FQ (IABkFQ)
  misc_feature 1: note = 5' 6-carboxyfluorescein (6-FAM)
agctgctattgttaccagtgacgcagct

SEQ ID NO: 25 - DNA, 28 bp, synthetic construct
  source 1..28: mol_type = other DNA
  misc_feature 28: note = 3' Iowa Black FQ (IABkFQ)
  misc_feature 1: note = 5' HEX (HEX is 2',4', 5',7', 1,4- Hexachlorofluorescein)
tggccagtgtattctgcacaggtggcca

SEQ ID NO: 26 - DNA, 601 bp, unidentified
  source 1..601: mol_type = other DNA
gttaacatgcattagaaaactaaatcttttgaaaagttgtagtatgccccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtactatgataaagttatctagaaataaaaaagcatacawttgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctagg

SEQ ID NO: 27 - DNA, 57 bp, synthetic construct
  source 1..57: mol_type = other DNA
agctgctattgttaccagtgacgcagctgaagctccacaatttggtgaattatcaat

SEQ ID NO: 28 - DNA, 57 bp, synthetic construct
  source 1..57: mol_type = other DNA
tggccagtgtattctgcacaggtggccagaagctccacaatttggtgaattatcaaa

SEQ ID NO: 29 - DNA, 29 bp, synthetic construct
  source 1..29: mol_type = other DNA
cactctagtactatatctgtcacatggta

SEQ ID NO: 30 - DNA, 28 bp, synthetic construct
  source 1..28: mol_type = other DNA
  misc_feature 28: note = 3' Iowa Black FQ (IABkFQ)
  misc_feature 1: note = 5' 6-carboxyfluorescein (6-FAM)
agctgctattgttaccagtgacgcagct

SEQ ID NO: 31 - DNA, 28 bp, synthetic construct
  source 1..28: mol_type = other DNA
  misc_feature 28: note = 3' Iowa Black FQ (IABkFQ3)
  misc_feature 1: note = 5' HEX (HEX is 2',4', 5',7', 1,4- Hexachlorofluorescein)
tggccagtgtattctgcacaggtggcca

SEQ ID NO: 32 - DNA, 27 bp, synthetic construct
  source 1..27: mol_type = other DNA
gctgctattgttaccagtgacgcagct

SEQ ID NO: 33 - DNA, 27 bp, synthetic construct
  source 1..27: mol_type = other DNA
ggccagtgtattctgcacaggtggcca